(19) United States
(12) Patent Application Publication
(10) Pub. No.: US 2011/0240042 A1
(43) Pub. Date: Oct. 6, 2011

(54) SPINAL IMMOBILIZATION

(75) Inventors: Matthew William Cooke, Coventry West Midlands (GB); Stuart Ward, Coventry West Midlands (GB)

(73) Assignee: THE UNIVERSITY OF WARWICK

(21) Appl. No.: 13/140,198

(22) PCT Filed: Dec. 16, 2009
(86) PCT No.: PCT/GB09/51721

§ 371 Date: Jun. 16, 2011

(30) Foreign Application Priority Data
Dec. 16, 2008 (GB) 0822886.8

(51) Int. Cl. A61F 5/37

(52) U.S. Cl. 128/870

(57) ABSTRACT

A spinal immobilisation apparatus has a harness for immovably securing to the torso of a person and a separate head support of collarless construction for mounting about the head of a person. Releasable connection allows the head support to be adjustably secured on the harness in relation to the shoulders and back of the wearer. The harness configuration allows ready access to the wearer's chest area. Elastication allows the cheat to expand during breathing. The head support is of open-faced, open-topped structure having rigid side walls hingedly connected to a rigid rear wall.

The present invention relates to techniques and apparatus for spinal immobilisation, more particularly, but not exclusively, to ‘collarless’ techniques and apparatus for spinal immobilisation.

Spinal immobilisation is essential for many victims of trauma with suspected neck injury, such as persons involved in severe road traffic accidents. Conventional spinal immobilisation techniques typically involve the use of a neck collar (commonly referred to as a “cervical collar”), which is designed to limit movement of a person's head and neck. However, conventional cervical collars suffer from several disadvantages, such as raised intracranial pressure due to pressure of the collar on the root of the wearer's neck and risk of aspiration of vomit due to limited allowable movement of the wearer's mouth caused by the collar. Other disadvantages include airway obstruction and restrictive ventilation. Some patients also experience marked claustrophobia and anxiety when immobilised using conventional cervical collars. Moreover, tests using magnetic resonance scanning have shown that some conventional cervical collars still allow for an unsuitable degree of movement of a wearer's neck.

There is need to improve or provide an alternative to conventional cervical collars.

It is an object of the invention to provide a spinal immobilisation apparatus which overcomes or substantially obviates one or more of the disadvantages set forth above.

According to a first aspect of the invention, there is provided a spinal immobilisation apparatus comprising a harness for immovably securing to the torso of a person and a head support of collarless construction for mounting about the head of a person, wherein the head support is securable to the harness for the purpose of supporting a wearer's head, to immobilise the wearer's neck against significant movement.

The term “collarless construction” is intended to mean that the head support does not define an annular support structure configured to sit around the neck of a wearer.

The apparatus is advantageous in that it provides an alternative to conventional cervical collars, wherein the wearer's neck can be immobilised against significant movement, by cooperation between the immovable harness and the head support secured thereto. Hence, the invention eliminates the need for a conventional cervical collar in trauma cases where neck immobilisation is required.

The head support is preferably configured for limiting movement of the wearer's head in three planes of movement, i.e. side-to-side movement, front-to-back movement (and vice versa), and rotational movement, once secured to the harness. For example, the head support preferably includes opposing side supports for contact with opposing sides of the wearer's head and a rear support for contact with the back of a person's head, wherein, in use, the side and rear supports combine to limit movement of the wearer's head in the three planes of motion referred to above.

The head support is preferably a collarless, open faced device, which is advantageous in reducing the risk of discomfort from claustrophobia, and permits ready access to the wearer's throat area, e.g. to allow for an emergency tracheostomy.

In preferred embodiments, the harness includes parts which extend over the wearer's shoulders, and the head support is preferably mounted on these parts. The apparatus is thus advantageous in that the head support may be effectively carried by the wearer's shoulders, reducing the risk of pressure to the root of the wearer's neck.

It will be understood that the head support is most preferably detachable from the harness, to enable the harness to be applied to a person independently of the head support. This is of particular advantage in confined spaces, such as in the event of a road traffic accident, in which the apparatus needs to be applied to a person trapped in the seat of a car, for example.

The head support is preferably movable between a released position relative to the harness (e.g. in which the head support is detached from the harness), wherein the wearer's neck is unsupported against significant movement, and a secured position (i.e. in which the head support is securably mounted on the harness), wherein the wearer's neck is immobilised against significant movement.

The apparatus is preferably configured to permit adjustment of the head support relative to the harness, in order for the apparatus to be adaptable to a range of sizes and configurations of wearer. In particular, the head support is preferably capable of multiple secured positions on the harness. In each of the secured positions, the head support is preferably mounted on parts of the harness extending over the shoulders of the wearer.

In preferred embodiments, the apparatus includes a releasable connection between the harness and the head support, by means of which the head support can be secured on the harness, for the purpose of supporting the wearer's head to immobilise the wearer's neck against significant movement. The releasable connection is preferably achieved using conventional ‘hook and eye’ fastener materials (also known as ‘hook and loop’ fastener materials, such as those marketed under the trade mark Velcro), provided between the harness and the head support, by means of which the head support can be releasably secured on the harness. However, other conventional reclosable or releasable fixture systems may be used for the releasable connection, for example an array of press studs, magnetic tape, mushroom type redo sable fastener materials (e.g. as marketed under the trade mark “Duotec”), ‘track’ or ‘rail’ and gimble arrangements, ‘Christmas tree’ clip and hole type arrangements, and high friction clamping arrangements. Equivalent systems and materials are within the scope of the invention and are not described here.

In each case, the releasable connection is preferably configured to enable the head support to be adjustably secured on the harness, for the purpose of supporting the wearer's head to immobilise the wearer's neck against significant movement. More particularly, it is preferred if the releasable connection permits movement of the head support from a first secured position on the harness to at least one further secured position on the harness, so that the apparatus is adaptable for different sizes and configuration of wearer.

The harness preferably includes an array of straps configured to be tightened so as to secure the harness on the torso of the wearer, so that the harness is substantially immovable on the torso, for providing a fixed datum on which to secure the head support. The harness preferably includes one or more quick release fasteners, e.g. a quick release buckle, to enable easy release of the harness from a tightened position on the wearer's torso.

In preferred embodiments, the harness includes a belt strap configured to pass around the waist or chest of a wearer, and shoulder straps configured to pass over a wearer's shoulders, wherein the shoulder straps are coupled with the belt strap and the straps are configured to be tensioned, for immovably securing the harness to the wearer's torso.

The harness preferably includes a quick release means for the belt strap.

In preferred embodiments, the belt strap is resiliently extensible, e.g. in order to permit the strap to increase in length, to facilitate expansion of a wearer's chest during breathing when the harness is otherwise immovably secured on the wearer's torso. For example, the belt strap may include or be coupled to an elastic or otherwise resiliently extensible section.

The harness preferably includes a back plate, more preferably a rigid back plate, configured to be positioned against a user's back. The back plate is advantageous in numerous applications, such as in road traffic accidents, e.g. wherein the back plate can be slid behind a wearer trapped in a vehicle seat before the harness is secured about the torso of the wearer.

The back plate may include a cushioning layer (e.g. of foam) intended to be positioned against the wearer's back, to improve wearer comfort. The outer surface of the back plate preferably includes a low friction coating, e.g. for assisting application of the back plate behind a person trapped in a vehicle seat, wherein the back plate is able to slide into position across the seat material.

The belt and shoulder straps are preferably carried by the back plate, e.g. passing through apertures in the back plate for securing the back plate in position on the wearer, in use.

In a preferred embodiment, the head support defines an open faced box structure having walls for mounting about the sides and rear of a wearer's head. The rear and side walls of the box structure are preferably at least 200 mm in height and preferably at least 140 mm in width, so as to be suitable for use with a wide range head sizes. The walls may be hingedly connected, to allow for adjustment of the relative positions of the side walls, e.g. for the purpose of clamping the wearer's head between the side walls, to limit neck movement.

The head support preferably includes a rear connector portion, more preferably in the form of a movable flap depending from the rear of the head support, which is adapted for releasable connection to the harness, more preferably to a rigid back plate of the harness, e.g. via conventional hook and eye type fastening materials (or equivalent releasable fastening systems) applied between the head support and the harness or back plate.

The head support preferably includes side connector portions, more preferably in the form of a movable flap depending from each side of the head support, adapted for releasable connection to the harness, more preferably to shoulder straps of the harness, e.g. via conventional hook and eye type fastening materials (or equivalent releasable fastening systems) applied between the head support and the harness or shoulder straps.

The rear and side connector portions of the head support are preferably of a size and dimension which permits adjustment of the head support relative to the harness across a range of sizes and configurations of wearer.

The side connector portions are preferably at least 40 mm in length and are more preferably in the region of 40-200 mm in length. The rear connector portion is preferably at least 75 mm in length and more preferably in the regions of 75-200 mm in length.

The head support preferably includes an adjustable strap configured to extend over a person's forehead and adapted to tighten the rear and side walls against the wearer's head.

The head support may include a chin strap, preferably adjustable, for engagement with a person's chin, to limit movement of the wearer's lower jaw.

According to another aspect of the invention, there is provided a spinal immobilisation technique involving a harness which is immovably secured to the torso of a person, and a head support of collarless construction which is mounted about the head of said person, wherein the head support is then secured to the harness for the purpose of supporting the wearer's head, to immobilise the wearer's neck against significant movement, e.g. to limit movement of the wearer's head in side-to-side movement, nodding movement and rotation.

The head support is preferably carried on the shoulders of the wearer. More particularly, the head support preferably includes lateral portions which are releasably connected to shoulder portions of the harness, in order to support the wearer's head against movement. In addition, the head support preferably includes a rear portion which is releasably connected to a rigid back pate of the harness, in order to further support the wearer's head against movement, e.g. to limit movement of the wearer's head in side-to-side movement, nodding movement and rotation.

The technique preferably involves the use of a removable head support defining an open faced box structure, including opposing side supports for contact with opposing sides of the wearer's head and a rear support for contact with the back of a person's head, wherein the side and rear supports are arranged to limit movement of the wearer's head in side-to-side movement, nodding movement and rotation, when positioned on the wearer and appropriately connected to the harness.

It will be understood that features of the harness and head supports described above may be of advantage in applications outside of the field of spinal immobilisation. Hence, the applicant reserves the right to apply for protection for the harness independently of the head support, and vice versa.

According to a further aspect of the invention, there is provided a harness for immovably securing to the torso of a person, the harness having one or more of the following features:
an array of straps configured to be tightened so as to secure the harness on the torso of the wearer, so that the harness is substantially immovable on the torso;
multiple fixture positions for a head support, so that a head support may capable of multiple secured positions on the harness, e.g. to allow adjustment of the head support relative to the harness, so as to be adaptable to a range of sizes and configurations of wearer;
parts which extend over a wearer's shoulders and which are adapted for releasably securing a head support thereto, e.g. including releasable fastener devices or materials arranged for cooperation with complimentary releasable fastener devices or materials provided on a head support intended for use therewith;
one or more quick release fasteners, e.g. a quick release buckle, to enable easy release of the harness from a tightened position on the wearer's torso;
a belt strap configured to pass around the waist or chest of a wearer, and shoulder straps configured to pass over a wearer's shoulders, wherein the shoulder straps are coupled with the belt strap and the straps are configured to be tensioned, for immovably securing the harness to the wearer's torso;
a resiliently extensible belt strap, to facilitate expansion of a wearer's torso during breathing when the harness is otherwise immovably secured on the wearer's torso;
a back plate, more preferably a rigid back plate, configured to be positioned against a user's back.

The harness is particularly suited for use as part of an apparatus for use in immobilising the spine of a wearer, e.g. in accordance with the first aspect of the invention.

According to a still further aspect of the invention, there is provided a head support of collarless construction for mounting about the head of a person, wherein the head support is configured for limiting movement of the wearer's head in three planes of movement, e.g. side-to-side movement, front-to-back movement (and vice versa), and rotational movement, in use.

For example, the head support preferably includes opposing side supports for contact with opposing sides of the wearer's head and a rear support for contact with the back of a person's head, wherein, in use, the side and rear supports combine to limit movement of the wearer's head in the three planes of motion referred to above.

The term “collarless construction” is intended to mean that the head support does not define an annular support structure configured to sit around the neck of a wearer.

The head support is preferably a collarless, open faced device, which is advantageous in reducing the risk of discomfort from claustrophobia, and permitting ready access to the wearer's throat area, e.g. to allow for an emergency tracheoptomy.

The head support is particularly suited for use as part of an apparatus for use in immobilising the neck of a wearer, e.g. in accordance with the first aspect of the invention.

The head support may comprise one or more of the following:
an open faced box structure having walls for mounting about the sides and rear of a wearer's head, wherein the rear and side walls of the box structure are preferably at least 200 mm in height and preferably at least 140 mm in width.
a rear portion, more preferably in the form of a movable flap depending from the rear of the head support, which is adapted for releasable connection to a harness, more preferably to a rigid back plate of a harness, e.g. via conventional hook and eye type fastening materials (or equivalent releasable fastening systems) applied between the head support and the harness or back plate.
side portions, more preferably in the form of a movable flap depending from each side of the head support, adapted for releasable connection to the a harness, more preferably to shoulder straps of a harness, e.g. via conventional hook and eye type fastening materials (or equivalent releasable fastening systems) applied between the head support and the harness or shoulder straps.
an adjustable strap configured to extend over a person's forehead and adapted to tighten the rear and side walls against the wearer's head.
a chin strap, preferably adjustable, for engagement with a person's chin, to limit movement of the wearer's lower jaw.

Other aspects and preferred features of the invention will be readily apparent from the following description of preferred embodiments, made by way of example only, with reference to the accompanying Figures, in which:

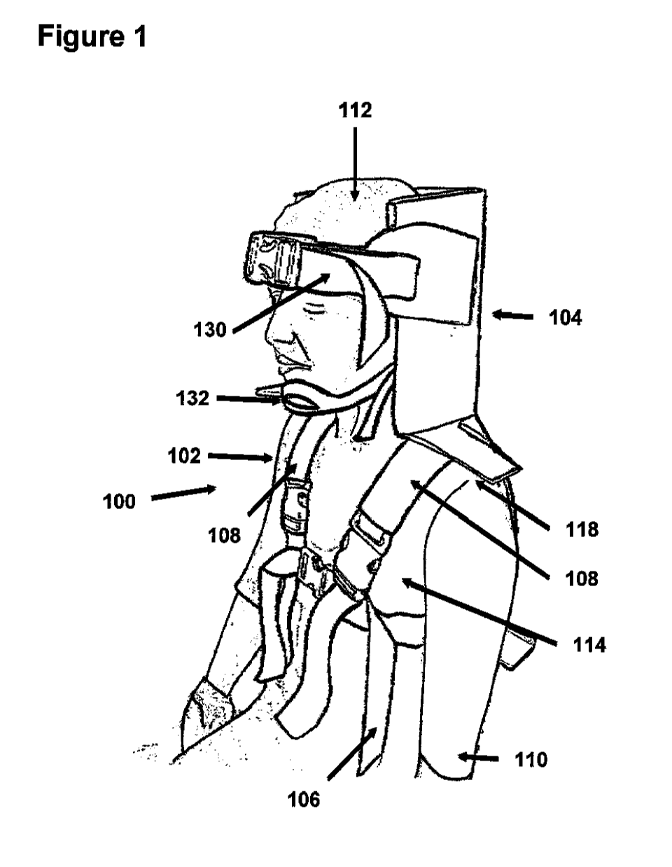

FIG. 1 is a schematic perspective view showing a spinal immobilisation apparatus in accordance with a preferred embodiment of the invention, secured in an operative position on a wearer;

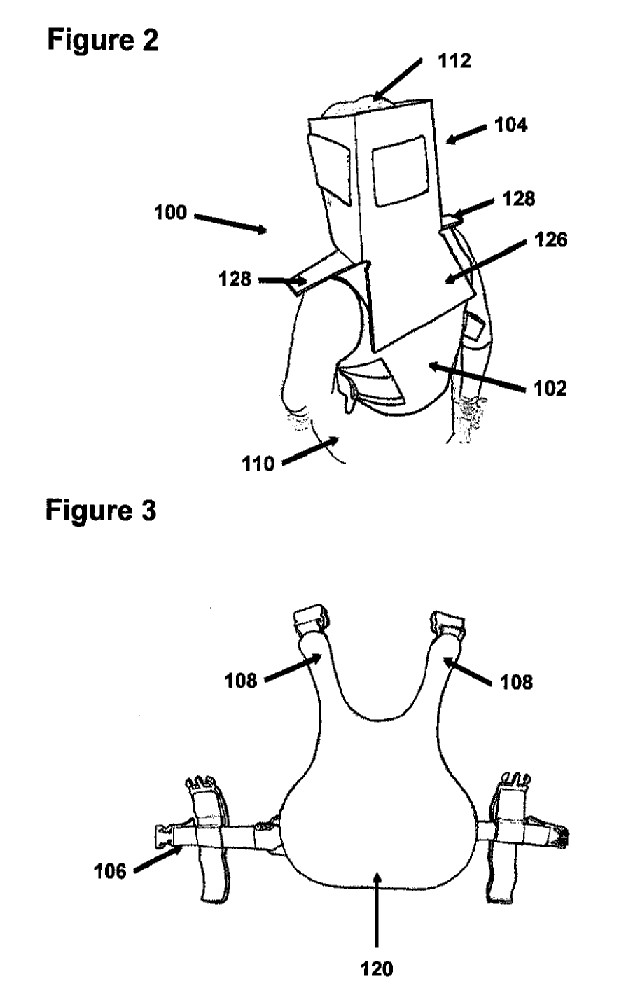

FIG. 2 is similar to FIG. 1, showing the spinal immobilisation apparatus from the rear;

FIG. 3 is a schematic view from the front of the chest harness in FIGS. 1 and 2, in a released position;

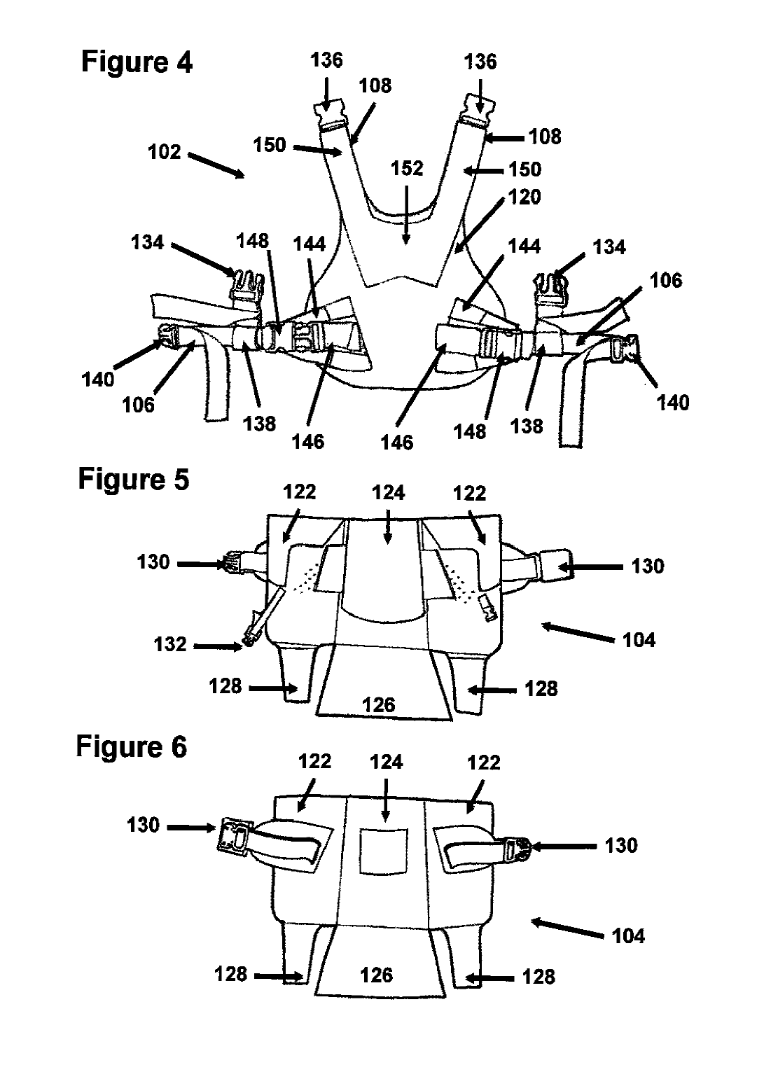

FIG. 4 is a schematic view from the rear of the chest harness in FIGS. 1 and 2, in a released position;

FIG. 5 is a schematic view from front of a preferred head support, in a released and open position;

FIG. 6 is a schematic view from rear of the head support in FIG. 5, in a released and open position.

Referring firstly to FIG. 1, a spinal immobilisation apparatus is indicated generally at 100, mounted on a wearer 110 (indicated in outline only). The apparatus 100 includes a chest harness 102 for mounting about the torso 114 of the wearer 110 and a head support 104 for mounting about the head 112 of the wearer 110.

As will be described in more detail below, the head support is securable to the chest harness for the purpose of supporting a wearer's head, to immobilise the wearer's neck against significant movement. The head support 104 is intended for limiting movement of the wearer's head 112 in three planes of movement, i.e. side-to-side movement, front-to-back movement (and vice versa), and rotational movement, in order to immobilise the wearer's neck, once coupled to the chest harness 102.

The apparatus is advantageous in that it provides an alternative to conventional cervical collars, wherein the wearer's neck can be immobilised against significant movement, by cooperation between the immovable harness and the head support secured thereto. The invention thus eliminates the need for a conventional cervical collar for victims of trauma with suspected neck injury, such as persons involved in severe road traffic accidents.

The head support 104 is of collarless construction. Hence, the head support 104 differs from conventional cervical collars in that it does not define a ring or other annular support structure configured to sit around the neck of a wearer 110. In this embodiment, the head support 104 defines an open-faced box-type structure for mounting about the sides and rear of a wearer's head 112. The box structure has opposing side walls separated by a rear wall, each wall being preferably at least 200 mm in height and preferably at least 140 mm in width, so as to be suitable for use across a wide range of head sizes.

It will be understood that the use of a collarless, open faced structure is advantageous in avoiding the application of pressure to the root of the neck, as well as in reducing the risk of discomfort from claustrophobia, and in permitting ready access to the wearer's throat area, e.g. to allow for an emergency tracheostomy. Moreover, it is possible to open the wearer's mouth whilst maintaining immobilization of the neck, whereas this is not possible using a conventional collar-type head support.

As will be described in more detail below, the head support 104 is releasably coupled to the chest harness 102. The releasable nature of the head support 104 enables the chest harness 102 to be fitted to the wearer 110 independently of the head support 104. As such, the head support 104 is movable between a released position, e.g. detached from the chest harness 102, in which the wearer's neck is unsupported against significant movement, and a secured position, i.e. securably mounted on the harness 102, in which the wearer's neck is supported against significant movement.

The secured position of the head support 104 is preferably adjustable relative the chest harness 102, to enable the apparatus 100 to be adaptable to a range of sizes and configurations of wearer 110.

The chest harness 102 includes an array of straps configured to be tightened so as to secure the chest harness 102 on the torso 114 of the wearer 110. Hence, in use, the chest harness 102 will be generally immovable on the torso 114 of the wearer 100, in order to provide a fixed datum on which to mount the head support 104.

In this embodiment, the chest harness 102 includes a belt strap 106 configured to pass around the waist or chest 116 of the wearer 110, and shoulder straps 108 configured to pass over the wearer's shoulders 118. The shoulder straps 108 are coupled with the belt strap 106. Both are configured to be tensioned, for immovably securing the harness 102 to the wearer's torso 114.

As illustrated, the belt strap 106 and shoulder straps 108 include conventional quick release click-type buckle fasteners and conventional length adjusting arrangements (for increasing or decreasing the tension in the straps), e.g. of the kind commonly used on other types of body harness or on conventional items of luggage. Their configuration and operation will be well known to the skilled person and so further description in unwarranted. Suitable equivalents within the scope of the invention will be readily apparent to the skilled person.

Referring to FIGS. 2 and 3, the chest harness 102 includes a back plate 120, preferably of rigid construction, which is intended to be positioned against the wearer's back. The belt strap 106 and shoulder straps 108 are coupled to the back plate 120.

The use of a rigid back plate is particularly advantageous. For example, it is suitable for sliding into position behind a wearer 110 trapped in a vehicle seat, before the chest harness 102 is secured about the torso 114 of the wearer 110. Moreover, once suitably tightened onto the torso 114, the rigid back plate 120 provides an inflexible datum onto which the head support 104 may be secured.

Referring to FIG. 4, it should be understood that a clip 134 which forms one part of the releasable fastener for the shoulder straps 108 (the other part 136 being provided at a distal end of the shoulder straps 108) is mounted on the belt strap 106 via a floating connection, indicated at 138, so as to be movable along the belt strap 106, as desired for the comfort of the wearer 110.

A further feature evident from FIG. 4 is a resilient aspect of the belt strap 106, which permits the wearer to expand his/her torso during normal breathing, when the chest harness 102 is otherwise immovably secured to the wearer's torso 114. The belt strap 106 has two end portions releasably connectable to one another via a conventional clip or buckle type connection 140. The belt strap 106 is carried by and passes through the back plate 120 and include an elastic section 144 which permits a degree of extension of the respective belt strap portion 106, to allow movement during normal breathing. In addition, a secondary elastic section 146 is carried by and passes through the back plate 120. The secondary elastic section 146 has two free ends, each of which are releasably coupled to a respective belt strap portion 106 (in this embodiment, via a conventional clip or buckle type fastener 148 of the kind used elsewhere on the apparatus 100), in order to limit the permissible degree of extension of the belt strap 106 during normal breathing. The secondary elastic sections 146 are adjustable in length in conventional manner, as are the belt strap portions 106.

In this embodiment, it can be seen that each shoulder strap 108 includes a covering of releasable fastener material 150 (e.g. a hook and eye type material) which also extends onto the upper region 152 of the back plate 120, for cooperation with sections of complimentary releasable fastener material on the head portion 104 of the apparatus 100.

Referring to FIGS. 5 and 6, a head support 104 (similar in construction to the head support from FIGS. 1 and 2) is configured for limiting movement of the wearer's head in three planes of movement, in order to immobilise the wearer's neck, i.e. side-to-side movement, front-to-back movement (and vice versa), and rotational movement, once coupled to the chest harness 102. In this embodiment, the head support 104 includes opposing side supports 122 for contact with opposing sides of the wearer's head 112 and a rear support 124 for contact with the back of the wearer's head 112.

In use, when the head support 104 is secured in place on the generally fixed chest harness 102, the side and rear supports 122, 124 cooperate with one another to limit movement of the wearer's head 112 in the three planes of motion referred to above.

It can be seen that the side and rear supports 122, 124 are provided by the side and rear walls of the head support 104, which define an open-faced, collarless box type structure for mounting about the head of a patient in the manner shown in FIGS. 1 and 2. The head support 104 is also of open-topped construction, for allowing access to the top of the wearer's skull.

A hinged connection between the side and rear walls, allows for adjustment of the relative position of the side walls, e.g. for the purpose of clamping the wearer's head 112 between the side supports walls. The side and rear walls are preferably of flat and rigid construction. However, moulding may be provided on the inside walls of the head support, for conforming to the shape of the respective side of wearer's head.

It should be immediately apparent that the head support 104 is mounted on parts of the harness 102 which extend over the shoulders 118 of the wearer 110.

In the illustrated embodiment, the head support 104 includes lateral portions, consisting of movable projections or flaps 128 extending from either side of the head support 104. These flaps 128 are intended for releasable connection to the chest harness 102. In particular, the flaps 128, are intended to be releasably secured to the shoulder straps 108 of the chest harness 102. This is preferably via the use of conventional hook and eye type fastening materials (or equivalent releasable fastening systems) applied between the flaps 128 and the shoulder straps 108.

Furthermore, the head support 104 includes a rear portion, in the form of a movable projection or flap 126 extending from the rear of the head support 104, which is also intended for releasable connection to the chest harness 102. In particular, the flap 126 is intended to be releasably secured to the rigid back plate 120 of the chest harness (see FIG. 2). Again, this may be via the use of conventional hook and eye type fastening materials (or equivalent releasable fastening systems) applied between the flap 126 and the back plate 120.

The flaps 126, 128 are preferably of a size and dimension which permits adjustment of the head support 104 relative to the chest harness 102 across a range of sizes and configurations of wearer. The underside of the flaps 126, 128 preferably include a covering of releasable fastener material (e.g. a hook and eye type material), for cooperation with complimentary releasable fastener material mounted on the chest harness 102.

Hence, it will be understood that the head support 104 is capable of multiple secured positions on the chest harness 102, in each of which the head support 104 is securably mounted on parts of the harness extending over the shoulders of the wearer. Additional support is provided by the connection between the head support 104 and the back plate of the harness 102.

By adjusting the affixed position of the side flaps 128 on the shoulder straps 108, a person fitting the apparatus 100 is able to push the side supports 122 into clamping engagement with the wearer's head 112, in order to restrict head movement.

The side flaps 128 are preferably at least 40 mm in length and are more preferably in the region of 40-200 mm in length. The rear flap 126 is preferably at least 75 mm in length and more preferably in the regions of 75-200 mm in length.

The head support 104 includes an adjustable strap 130 configured to extend across the wearer's forehead and adapted to tighten the side and rear supports 122, 124 against the wearer's head 112.

The head support 104 also includes an adjustable chin strap 132, configured to limit movement of the wearer's lower jaw.

The apparatus 100 is advantageous in that it can be fitted to a patient in situ, e.g. in a vehicle after a road traffic accident, so that the patient's neck is immobilised against significant movement prior to lifting the patient to an ambulance or other location (the highest risk period). The apparatus 100 also allows a level of access to the patient's neck not previously possible using conventional cervical collars.

The use of shoulder straps connecting to a belt strap provides an “open chest” arrangement of the harness straps, which allows access to the user's chest, e.g. for relieving a collapsed lung, etc. Furthermore, the strap arrangement, allows for less restriction of chest movement than conventional chest strap configurations. This is enhanced in those embodiments which incorporate elastication in the belt strap configuration.

In the most preferred embodiments, head support is removably and adjustably mounted on shoulder portions of the harness, in which case it is preferred if the head support is configured to projects rigidly from the shoulder portions, in use, for immobilising the wearer's head and neck.

Although not illustrated, the back plate may include a cushioning layer (e.g. of foam) intended to be positioned against the wearer's back, to improve wearer comfort. The outer surface of the back plate may include a low friction coating, e.g. for assisting application of the back plate behind a person trapped in a vehicle seat, wherein the back plate is able to slide into position across the seat material.

In each of the embodiments, the releasable connection between the head support 102 and the chest harness 104, e.g. the cooperation between the hook and eye-type fastener materials, is preferably suitable to remain immovably secure during movement of the patient, e.g. during transfer of a patient from an accident scene to an ambulance or air helicopter.

The releasable connection between the chest harness and the head support, by means of which the head support can be secured on the chest harness, for the purpose of supporting the wearer's head to immobilise the wearer's neck against significant movement, is preferably achieved using conventional ‘hook and eye’ fastener materials (also known as ‘hook and loop’ fastener materials, such as those marketed under the trade mark Velcro), provided between the chest harness and the head support. However, other conventional reclosable or releasable fixture systems may be used for the releasable connection, for example an array of press studs, magnetic tape, mushroom type reclosable fastener materials (e.g. as marketed under the trade mark “Duotec”), ‘track’ or ‘rail’ and gimble arrangements, ‘Christmas tree’ clip and hole type arrangements, and high friction clamping arrangements. Equivalent systems and materials are within the scope of the invention and are not described here. In each case, the releasable connection is preferably configured to enable the head support to be adjustably secured on the chest harness, for the purpose of supporting the wearer's head to immobilise the wearer's neck against significant movement. More particularly, it is preferred if the releasable connection permits the movement of the head support from a first secured position to at least one further secured position on the chest harness, so as to be adaptable for different sizes and configuration of wearer.

The apparatus is preferably radio lucent (to allow x-ray and CT scanning of the patient, when wearing the apparatus), magnetically inert (to allow MR scanning, when wearing the apparatus), cleanable (to allow disinfection and blood decontamination, after use) and/or may be made of bacteriostatic materials (to reduce the risk of infection transmission). The apparatus is preferably devoid of ferrous compounds.

It will be understood that features of the chest harness and head supports described above may be of advantage in applications outside of the field of spinal immobilisation. For example, it is envisaged that the chest harness described herein could be used independently as a harness which could be adapted for carrying children or for use in supporting a rucksack etc. Hence, the applicant reserves the right to apply for protection for aspects of the chest harness independently of the head support, and vice versa.

1-35. (canceled)

36. A spinal immobilization apparatus comprising a harness for immovably securing to the torso of a person and a head support of collarless construction for mounting about the head of a person, wherein the head support is separate from the harness and is adapted to be adjustably secured on the harness, so as to be movable between a detached position relative to the harness, and a plurality of secured positions on the harness in relation to the shoulders and back of the harness wearer, in which the wearer's neck is immobilized against significant movement.

37. Apparatus according to claim 36, including a releasable connection between the harness and the head support, by means of which the head support can be secured on the harness, for the purpose of supporting the wearer's head to immobilize the wearer's neck against significant movement.

38. Apparatus according to claim 36 wherein the harness includes shoulder portions configured to be positioned over the shoulders of the wearer and the head support is removably and adjustably mounted on said shoulder portions.

39. Apparatus according to claim 38 wherein the head support includes lateral portions adapted for releasable connection to shoulder portions of the harness.

40. Apparatus according to claim 36 wherein the harness includes a rigid back plate configured to be positioned against a user's back.

41. Apparatus according to claim 40 wherein the head support includes a rear portion adapted for releasable connection to the back pate of the harness.

42. Apparatus according to claim 37 wherein the releasable connection is achieved using reclosable fastener-type materials provided between the support and the harness.

43. Apparatus according to claim 36 wherein the harness includes an array of straps configured to be tightened for securing the harness on the torso of the wearer, so that the harness is substantially immovable on the torso, for to providing a generally fixed datum on which to secure the head support, and includes elastication for allowing chest expansion during breathing when the harness is otherwise immovably secured on the user's torso

44. Apparatus according to claim 36 wherein the harness includes a belt strap configured to pass around the waist or chest of a wearer, and shoulder straps configured to pass over a wearer's shoulders, and has an ‘open chest’ arrangement of the harness straps, for allowing ready access to the user's chest area.

45. Apparatus according to claim 36 wherein the head support defines an open faced box structure having opposing side walls of rigid construction separated by a rear wall of rigid construction, for mounting about the sides and rear of a wearer's head.

46. Apparatus according to claim 45 wherein the head support is further of open-topped structure, for allowing access to the top of the user's head.

47. Apparatus according to claim 45, including a hinge between the rear wall and each side wall, to allow for adjustment of the position of the side walls relative to the rear support.

48. Apparatus according to claim 36 wherein the head support includes opposing side supports for contact with opposing sides of the wearer's head and a rear support for contact with the back of a person's head, wherein, in use, the side and rear supports combine to limit movement of the wearer's head in the three planes of motion.

49. Apparatus according to claim 48 wherein the head support is configured to allow for adjustment of the position of the side supports relative to the rear support, for the purpose of clamping the wearer's head between the side supports to limit neck movement.

50. Apparatus according to claim 48 wherein the side and rear supports are of rigid construction, for preventing flexing of the supports.
